(19) United States
(12) Patent Application Publication
(10) Pub. No.: US 2011/0097427 A1
(43) Pub. Date: Apr. 28, 2011

(54) Molecular Targets and Dietary Modulators of Exercise-Induced Muscle Damage

(76) Inventors: Shyam Ramakrishnan, Whittier, CA (US); Aida Briseno, Fullerton, CA (US); Mary A. Murray, Irvine, CA (US); Kerry Grann, Lansing, MI (US)

(21) Appl. No.: 12/912,508

(22) Filed: Oct. 26, 2010

Related U.S. Application Data
(60) Provisional application No. 61/255,359, filed on Oct. 27, 2009.

(51) Int. Cl. A61K 36/738; A61K 36/87; A61P 21/00

(52) U.S. Cl. 424/765; 424/766

(57) ABSTRACT

Dietary supplement compositions include an adaptogenic agent, an anti-inflammation agent, and an anti-oxidant. Methods for using dietary supplement compositions include (i) inhibiting, decreasing, and/or preventing delayed onset of muscle soreness (DOMS); (ii) inhibiting, decreasing, and/or preventing exercise-induced muscle damage; and/or (iii) modulating the expression of genes that are correlated with exercise-induced muscle damage.

### RELATED APPLICATION

This application claims the benefit of U.S. Provisional Application No. 61/255,359, filed Oct. 27, 2009, the entire contents of which are hereby incorporated by reference.

### TECHNICAL FIELD

The present teachings relate generally to the field of dietary supplement compositions, and to methods useful for the modulation of gene expression and for the inhibition, decrease, and/or prevention of exercise-induced muscle damage.

### BACKGROUND

It is known that exercise can induce muscle damage and inflammation depending on the exercise mode and duration. Exercise with a large eccentric component (lengthening of a muscle that is actively developing tension) produces the greatest muscle fiber damage, inflammation, delayed-onset muscle soreness (DOMS) and various functional deficits.

When myofibrils within a muscle fiber are stretched during contraction, some sarcomeres are more resistant to stretching than others. Consequently, the weaker sarcomeres absorb more of the stretch and, depending on the length-tension ratio, these sarcomeres become weaker until there is little or no overlap between the myofilaments. During repeated eccentric contractions, first the weak and then the stronger sarcomeres are progressively overstretched. During the muscle relaxation phase, the myofilaments of overstretched sarcomeres may fail to reconnect, resulting in disrupted sarcomeres. This structural disruption can spread to adjacent areas of the muscle, and can ultimately lead to damage to the membranes of the sarcoplasmic reticulum, transverse tubules or the sarcolemma. At the same time, excitation-contraction coupling is disrupted, and Ca²⁺ moves freely into the sarcoplasm where it activates proteolytic pathways related to muscle fiber degradation and repair (Luke, Am. J. Clin. Nutr., 2000, 72(suppl), 624S-36S). This process appears to produce some of the symptoms associated with muscle damage, including loss of muscle function, DOMS, and plasma membrane damage. Skeletal muscle adapts to exercise-induced damage, such that there is less muscle damage and soreness when eccentric exercise (ECC) using the same muscles is repeated up to 6 months after an initial bout. However, the precise mechanisms contributing to this repeated bout effect remain unclear (Proske and Allen, Exerc. Sport Sci. Rev., 2005, 33, 98-104).

The immune system plays a role in the degeneration and regeneration process of muscle and surrounding connective tissue after exercise-induced muscle damage. Briefly, neutrophils are rapidly mobilized into the circulation after exercise, and soon invade the damaged muscle tissue. Natural killer cells and lymphocytes are also mobilized, and anti-inflammatory cytokines are released into the systemic circulation during and immediately after eccentric exercise. Within one day after exercise, neutrophils are replaced in damaged muscle tissue by macrophages, and pro-inflammatory cytokines are produced in muscle. These inflammatory responses are important for the regulation of the acute-phase response and removal of fragments of damaged muscle after eccentric exercise.

There is a need to identify and characterize the genes, molecular targets, and pathways involved in the body's response to exercise. If these targets and/or pathways are differentially regulated with exercise, it would be beneficial to discover agents (for example, nutraceutical supplements) that can be administered to target the above mechanism(s).

### BRIEF SUMMARY

The scope of the present invention is defined solely by the appended claims, and is not affected to any degree by the statements within this summary.

In some embodiments, compositions comprise a combination of: (i) one or more adaptogenic agents selected from the group consisting of Rhodiola extract and Ashwagandha extract; (ii) one or more anti-inflammation agents selected from the group consisting of rose hips extract and grape seed extract; and (iii) one or more anti-oxidants selected from the group consisting of astaxanthin extract and prickly pear extract. The compositions may include about 10 to about 1000 mg of Rhodiola extract, about 30 to about 3000 mg of rose hips extract, and about 0.1 to about 100 mg of astaxanthin extract. In some embodiments, the compositions may include about 200 mg of Rhodiola extract, about 600 mg of rose hips extract, and about 4 mg of astaxanthin extract. In some embodiments, the compositions may include about 1 to about 2000 mg of Ashwagandha extract, about 10 to about 3000 mg of grape seed extract, and about 20 to about 5000 mg of prickly pear extract. In some embodiments, the compositions may include about 125 mg of Ashwagandha extract, about 200 mg of grape seed extract, and about 500 mg of prickly pear extract.

The compositions may modulate the expression of one or more genes, including but not limited to: PPARα (peroxisome proliferative activated receptor, alpha); PPARδ (peroxisome proliferative activated receptor, delta); IRF5 (Interferon regulatory factor 5); PLAUR (plasminogen activator, urokinase receptor); RSU1 (Ras suppressor protein 1); CEBPD (CCAAT/enhancer binding protein delta); IFI16 (interferon, gamma-inducible protein 16); TNNT2 (troponin-T type 2); GJA1 (gap junction protein, alpha-like); and SCN3B (sodium channel, voltage-gated, type III, beta). The modulation of gene expression may be up-regulation of gene expression. Alternatively, the modulation of gene expression may be down-regulation of gene expression.

The compositions in accordance with the present teachings may further include excipients. The compositions may inhibit, decrease, or prevent eccentric exercise-induced muscle damage in subjects.

Methods are disclosed for inhibiting, decreasing, or preventing the symptoms of DOMS in subjects. The methods include administering to the subjects compositions in accordance with the present teachings.

Methods are disclosed for inhibiting, decreasing, or preventing the symptoms of exercise-induced muscle damage in subjects. The methods include administering to the subjects compositions in accordance with the present teachings. The exercise-induced muscle damage may be eccentric exercise-induced muscle damage.

Dietary supplements for modulating the expression of one or more genes are disclosed. The genes that may be modulated by the dietary supplements include but are not limited to: PPARα (peroxisome proliferative activated receptor, alpha); PPARδ (peroxisome proliferative activated receptor, delta); IRF5 (Interferon regulatory factor 5); PLAUR (plasminogen activator, urokinase receptor); RSU1 (Ras suppressor protein 1); CEBPD (CCAAT/enhancer binding protein delta); IFI16 (interferon, gamma-inducible protein 16); TNNT2 (troponin-T type 2); GJA1 (gap junction protein, alpha-like); and SCN3B (sodium channel, voltage-gated, type III, beta). The dietary supplements may include one or more excipients and a combination of: (i) one or more adaptogenic agents selected from the group consisting of Rhodiola extract and Ashwagandha extract; (ii) one or more anti-inflammation agents selected from the group consisting of rose hips extract and grape seed extract; and (iii) one or more anti-oxidants selected from the group consisting of astaxanthin extract and prickly pear extract. In some embodiments, the dietary supplements may include about 10 to about 1000 mg of Rhodiola extract, about 30 to about 3000 mg of rose hips extract, and about 0.1 to about 100 mg of astaxanthin extract. The dietary supplements may include about 200 mg of Rhodiola extract, about 600 mg of rose hips extract, and about 4 mg of astaxanthin extract. In some embodiments, the dietary supplements may include about 1 to about 2000 mg of Ashwagandha extract, about 10 to about 3000 mg of grape seed extract, and about 20 to about 5000 mg of prickly pear extract. The dietary supplements may include about 125 mg of Ashwagandha extract, about 200 mg of grape seed extract, and about 500 mg of prickly pear extract.

Methods of modulating the expression of one or more genes correlated with eccentric exercise-induced muscle damage in subjects are disclosed. The methods include administering dietary supplements in accordance with the present teachings to the subjects. The modulation of gene expression in the subjects may be up-regulation of gene expression. Alternatively, the modulation of gene expression in the subjects may be down-regulation of gene expression.

### BRIEF DESCRIPTION OF THE DRAWINGS

FIG. 1 is a graph showing knee extension isometric force for a group receiving the placebo.

FIG. 2 is a graph showing knee extension isometric force for a group receiving supplement 1 (a combination of Rhodiola, rose hips, and astaxanthin)—a composition that is further described below.

FIG. 3 is a graph showing knee extension isometric force for a group receiving supplement 2 (a combination of Ashwagandha, grape seed, and prickly pear)—a composition that likewise is further described below.

### DETAILED DESCRIPTION

Nutrition supplements and, more specifically, sports nutrition supplements are described that improve muscle strength, performance, and/or recovery in multiple applications including but not limited to reduction of symptoms or maintenance of musculoskeletal disorders (MSDs) or musculoskeletal degenerative diseases or impairments.

Surprisingly, it has been discovered that novel combinations of various adaptogenics, anti-inflammatory agents, and antioxidants, can: (1) inhibit, decrease, and/or prevent DOMS; (2) inhibit, decrease, and/or prevent exercise-induced muscle damage; and/or (3) modulate (upregulate and/or downregulate) the expression of genes that may be correlated with exercise-induced muscle damage.

In one aspect, the present teachings relate to targeting of three mechanisms associated with DOMS: (i) inflammation; (ii) stress; and (iii) oxidation. These three mechanisms were targeted using novel combinations of anti-inflammatory, anti-stress (adaptogenic), and anti-oxidative ingredients. In one aspect of the present teachings, the identification and correlation of gene expression with measured improvement in DOMS in a human subject population is a novel and groundbreaking approach for designing supplements and identifying ingredients for this application.

The terms “dietary supplement”, “nutritional supplement”, “food supplement” or simply “supplement” refer to a preparation intended to provide nutrients when administered to a subject.

The term “adaptogen” is used herein to refer to a product that may increase the body's resistance to stress, trauma, anxiety, and/or fatigue. In the past, adaptogens have been called rejuvenating herbs, qi tonics, rasayanas, and/or restoratives. Polysaccharaides are a common constituent in many adapotogens that are believed to be involved in immune system stimulation. Adaptogens may be considered as having energy property, anti-oxidant property, or some other property including anti-nitrosative stress, etc. Adaptogens typically contain antioxidants but antioxidants are not necessarily adaptogens and that is not proposed to be their primary mode of action. A typical functional definition of an adaptogen is: 1) an adaptogen is nontoxic to the recipient; 2) an adaptogen produces a nonspecific response in the body—an increase in the power of resistance against multiple stressors including physical, chemical, or biological agents; 3) an adaptogen has a normalizing influence on physiology irrespective of the direction of change from physiological norms caused by the stressor. Under this definition, adaptogens would be nontoxic in normal doses, produce a general defensive response against stress, and have a normalizing influence on the body (Winston, David & Maimes, Steven. Adaptogens: Herbs for Strength, Stamina, and Stress Relief, Healing Arts Press: 2007). Without wishing to be bound by a particular theory or to in any way limit the scope of the appended claims or their equivalents, it is presently believed that that constituents possibly common to adaptogens are: 1) triterpenes (mevalonate pathway), including but not limited to: triterpenoid saponins (e.g., dammarane triterpene saponins, cucurbitacins); phytosterols (e.g., beta-sitosterol); and phytoecdysteroids (e.g., 20-ecdysone, turkesterone); 2) phenylpropanes (shikimate pathway), including but not limited to: flavonoids (glucopyranosides, prenylated flavonoids, flavan glycosides); lignans (schizandrin, sesamin, syringaresinol); and 3) oxylipins (acetate pathway), including but not limited to hydroxylated fatty acids (e.g., octadecadienoic acid). (Panossian, Natural Pharmacy, 2003, 7(4), 1:19-20).

Reference to adaptogens is meant to also include extracts of adaptogens (adaptogen extracts). Adaptogens (or adaptogen extracts) may be commercially obtained from various sources. In addition, adaptogen extracts (extracts of adaptogens) may be obtained using any of the extraction techniques known in the art.

It is contemplated that a variety of adaptogens (adaptogen agents) may be used in accordance with the present teachings. The adaptogens may be natural or synthetic. Examples of suitable adaptogens include, but are not limited to, the types of adaptogenic herbs described below as well as any combinations thereof.

1) Rhodiola rosea (“golden root”). Rhodiola is an adaptogenic herb that protects against stress-related fatigue and “burnout”; increases mental clarity; and offers immune and blood sugar support. An added benefit of Rhodiola is its antidepressant and antianxiety effects. Rhodiola extract may be commercially obtained from various sources, e.g. from PoliNat, Las Palmas, Spain, or from National Bioscience Corporation, Chester, N.Y.

2) Ashwagandha—Withania somnifera, also known as Indian ginseng, Winter cherry, Ajagandha, Kanaje Hindi, Ayamodakam in Malayalam and Samm Al Ferakh, is a plant in the Solanaceae family. Ashwagandha extract may be commercially obtained from various sources, e.g. from NutraGenesis, Brattleboro, Vt.

3) Astragalus root (Astragalus membranaceus). Astragalus aids in the body's natural ability to adapt to stress, bolstering the immune system so the subject stays well while helping to regulate normal blood sugar levels and alleviate insulin resistance.

4) Cordyceps (Cordyceps sinensis). Cordyceps is a fungus that can slow aging and take a load off the adrenals by supporting the immune system, balancing the inflammatory response, and helping to stabilize blood sugar.

5) Eleuthero (Eleutherococcus senticosus, formerly called Siberian ginseng). Eleuthero is an adaptogenic herb that can help protect against the negative effects of stress, while decreasing fatigue, enhancing mental clarity, helping to balance blood sugar, and even perhaps supporting bone remodeling as well.

6) Licorice root (Glycyrrhiza glabra). Licorice root may support adrenal balance and increase energy and endurance.

7) Asian ginseng (Panax ginseng). Asian ginseng is an adaptogen that exhibits anti-carcinogenic and anti-oxidant properties. It is believed to improve circulation, increase blood supply, revitalize and aid recovery following illness, and/or stimulate the body. Asian ginseng extract may be commercially obtained from various sources, e.g. from Draco Natural Products, San Jose, Calif.

The terms “anti-inflammatory agents”, “anti-inflammation agents”, or “anti-inflammatories” are used herein to refer to compounds that reduce inflammation. Anti-inflammatory drugs make up about half of analgesics, remedying pain by reducing inflammation.

Reference to anti-inflammatories or anti-inflammatory agents is meant to also include extracts of anti-inflammatories (extracts of anti-inflammatory agents; anti-inflammatories extracts). Anti-inflammatories (or extracts of anti-inflammatories) may be commercially obtained from various sources. In addition, anti-inflammation extracts (extracts of anti-inflammatory agents) may be obtained using any of the extraction techniques known in the art.

It is contemplated that a variety of anti-inflammatory agents (anti-inflammatories) may be used in accordance with the present teachings. The anti-inflammatory agents may be natural or synthetic. Examples of suitable anti-inflammatories include, but are not limited to, the types of anti-inflammatory agents described below as well as any combinations thereof.

1) Rose hips, the pomaceous fruit of the rose plant, which typically is red-to-orange, but might be dark purple-to-black in some species. Rose hips extract may be commercially obtained from various sources, e.g. from Plantextrakt GmbH & Co. KG, Vestenbergsgreuth, Germany, or from Nature's Answer, Hauppauge, N.Y.

2) Grape seeds. Grape seeds and grape seed extracts, which include a variety of polyphenols. Grape seed extract may be commercially obtained from various sources, e.g. from B & D nutritional Ingredients, Inc., Vista, Calif., or from Polyphenolics, Madera, Calif.

3) Bioflavonoids, also called flavones or flavonoids, include compounds such as quercetin, epicatechin, and oligomeric proanthocyanidins (OPCS). One example is pine bark extract (Pycnogenol).

4) Boswellia (Boswellia serrata). Also known as Indian frankincense, apparently it may switch off key cell signals and pro-inflammatory mediators known as cytokines in the inflammatory cascade.

5) Ginger (Zingiber officinalis). Ginger may share properties with conventional over-the-counter and prescription NSAIDs (non-steroidal anti-inflammatory drugs), in that it suppresses the synthesis in the body of the pro-inflammatory molecules known as prostaglandins—with few if any side effects. Ginger extract may also inhibit or deactivate genes that encode the molecules involved in chronic inflammation.

6) Turmeric (Curcuma longa), an ancient culinary spice native to Southeast Asia, is also known as cucurmin. It is a mild COX-2 inhibitor, but works differently from the prescription-strength drugs that can increase risk of myocardial infarction or stroke. It seems to inhibit joint inflammation by preventing the production of prostaglandins and activation of inflammation-regulating genes through its effects on cell-signaling.

The term “antioxidant” is used herein to refer to a molecule capable of slowing or preventing the oxidation of other molecules. Antioxidants can often be reducing agents such as thiols, ascorbic acid or polyphenols (Sies, Exp. Physiol., 1997, 82, 291-295).

Reference to antioxidants is meant to also include extracts of antioxidants (antioxidant extracts). Antioxidants (or antioxidant extracts) may be commercially obtained from various sources. In addition, antioxidants extracts (extracts of antioxidants) may be obtained using any of the extraction techniques known in the art.

It is contemplated that a variety of antioxidants may be used in accordance with the present teachings. The antioxidants may be natural or synthetic. Examples of antioxidants include, but are not limited to, the types of antioxidants described below as well as any combinations thereof.

1) Astaxanthin—a carotenoid, classified as a xanthophyll, fat/oil-soluble pigment. Astaxanthin can be found in microalgae, yeast, salmon, trout, krill, shrimp, crayfish, crustaceans, and the feathers of some birds. Astaxanthin extract may be commercially obtained from various sources, e.g. under the name of AstaREAL®P2 AF from Fuji Health Sciences, Burlington, N.J.

2) Prickly pear—Opuntia cacti. Prickly pear extract may be commercially obtained from various sources, e.g. under the name of Cacti-Nea Instant from Bio Serae Laboratories, Bram, France.

3) Vitamins and vitamin-like substances, for example vitamin C and/or vitamin E. Additional examples of vitamins include, but are not limited to, vitamin B2, vitamin C, vitamin E, and the vitamin-like coenzyme Q10.

4) Minerals. A variety of minerals have antioxidant properties.

5) Herbs. Examples include, but are not limited to, bilberry, aloe vera, green tea, turmeric, ginkgo, grape seed or pine bark extracts, milk thistle, and cascara sagrada also help protect the body from health problems caused by oxidants. Many fruits also contain antioxidants like mangosteen. Other herbals may include, for example, rosemary, sage, oregano, thyme, ginger, summer savory, black pepper, red pepper, clove, marjoram, basil, peppermint, spearmint, common balm, fennel, parsley, cinnamon, cumin, nutmeg, garlic, coriander, etc.

6) Coenzymes (cofactors for enzymes or enzyme complexes). An example of an antioxidant coenzyme is lipoic acid.

7) Peptides (for example, dipeptides, tripeptides, tetrapeptides, etc.). An example of an antioxidant tripeptide is gluthatione.

Although each of the extracts used in accordance with the present teachings is commercially available, there are numerous extraction methods that can be used to produce an extract in accordance with the present teachings without commercially purchasing the extract. Some examples of extraction methods that can be used to produce an extract in accordance with the present teachings are described below. Other examples are known and are described in various publications and patents. The extraction methods described more fully below are exemplary and one of ordinary skill in the art will appreciate that other extraction techniques and methods may be used to obtain an extract useful in accordance with the present teachings.

Extracts used in accordance with the present teachings may be from a variety of sources, including different varieties and species. For example, grape seeds from grapes of any color or variety may be used to obtain a grape seed extract. In addition, any of the parts of a plant may be extracted, including the fruit, peel, seeds, stem, leaves, roots, bark, rhizome, runner, and the like.

In one example, an extract may be obtained simply by a water extraction process. Such a process may include heating, for example grape seeds, to produce a turbid liquid which is then filtered to separate the liquid from the seeds. The liquid may then be filtered, purified, and dehydrated prior to spray drying, to provide a dried grape seed extract.

In another example, an extract useful in the unique compositions in accordance with the present teachings might be obtained using an organic solvent extraction technique.

In another example, solvent sequential fractionation may be used to obtain an extract useful in the unique compositions in accordance with the present teachings. For example, using this technique, a grape seed extract could be obtained by sequentially extracting grape seeds with hexane, ethyl acetate, ethanol, and hydro-ethanol. The extracts obtained after each step (fractions) of the sequence will contain chemical compounds in increasing order of polarity similar to the solvents used for extracting them. The fractions are dried to evaporate the solvents, resulting in an extract of grape seed. Those of skill in the art will appreciate that many other solvents can be used in practicing solvent sequential fractionation extraction.

Total hydro-ethanolic extraction techniques might also be used to obtain an extract useful in the unique compositions in accordance with the present teachings. Generally, this is referred to as a lump-sum extraction. The extract generated in this process will contain a broad variety of phytochemicals present in the extracted material including fat and water solubles. Following collection of the extract solution, the solvent will be evaporated, resulting in the extract.

Total ethanol extraction may also be used in accordance with the present teachings. This technique uses ethanol, rather than hydro-ethanol, as the solvent. This extraction technique generates an extract that may include fat soluble and/or lipophilic compounds in addition to water soluble compounds.

Another example of an extraction technique that might be used to obtain an extract useful in accordance with the present teachings is supercritical fluid carbon dioxide extraction (SFE). In this extraction procedure the material to be extracted is not exposed to any organic solvents. Rather, the extraction solvent is carbon dioxide, with or without a modifier, in supercritical conditions (>31.3° C. and >73.8 bar). Those of skill in the art will appreciate that temperature and pressure conditions can be varied to obtain the best yield of extract. This technique generates an extract of fat soluble and/or lipophilic compounds, similar to the total hexane and ethyl acetate extraction technique described above.

Those of skill in the art will appreciate that there are many other extraction processes, both known in the art and described in various patents and publications, that can be used to obtain extracts in accordance with the present teachings. For example, the extraction procedures described in the following references, which are incorporated herein by reference, could be used in accordance with the present teachings: Murga et al., J. Agric Food Chem., 2000, 48, 3408-3412; Hong et al., Nat. Prod. Lett., 2001, 15, 197-204; Ashraf-Khorassani et al., J. Agric Food Chem., 2004, 52, 2440-2444.

In one aspect, the present teachings are directed to the development of better molecular understanding of muscle fatigue. Thus, in one aspect, the present teachings are related to the identification of genes that are modulated in skeletal muscle after exercise, and in particular after eccentric exercise. For example, the modulation of genes may include upregulation of the expression of the genes in skeletal muscle after eccentric exercise. Alternatively, the modulation of genes may include downregulation of the expression of the genes in skeletal muscle after eccentric exercise. Thus, in some embodiments, the identification and optionally modulation of genes that are upregulated and/or genes that are downregulated in skeletal muscle after eccentric exercise are contemplated.

Eccentric exercise may be practiced as known in the art (Proske and Morgan, J. Physiol., 2001, Dec. 1, 537(Pt 2), 333-345). Eccentric exercise may be practiced by instrumentation-based measurement of strength using the devices and protocols of Biodex Medical Systems, Inc., Shirley, N.Y. (e.g., Biodex Multi-Joint system PRO, Biodex Multi-Joint system JPN, and/or Biodex Multi-Joint system w/o CMP), the descriptions of which are herein incorporated by reference.

In another aspect, the present teachings are related to the effects of administration of nutritional supplements (or dietary supplements, or simply supplements) on the modulation of the expression of genes that are correlated with exercise, and in particular with eccentric exercise. For example, some of these genes may be upregulated in skeletal muscle after eccentric exercise. Alternatively, some of these genes may be down-regulated in skeletal muscle after eccentric exercise. When nutritional supplements in accordance with the present teachings are administered to subjects, e.g. before and/or during and/or after exercise, the administered nutritional supplements may modulate the expression of one or more genes whose expression in skeletal muscle is associated with eccentric exercise.

In another aspect, the present teachings relate to the identification of compositions that can reduce muscle damage caused by exercise, and in particular to the identification of compositions that can reduce muscle damage caused by eccentric exercise. Thus, in some embodiments, the present teachings relate to compositions such as dietary supplements (or simply supplements) that can reduce muscle damage caused by eccentric exercise.

In another aspect, the present teachings are related to a well-known phenomenon in sports nutrition, DOMS, for which it is believed there are no currently known, effective, and clinically proven supplements. DOMS is represented by symptoms including strength loss, pain, muscle tenderness, stiffness, and swelling. Clinical studies related to DOMS measure one or more of strength loss, pain, and muscle gene expression before and after supplement intake. One or more of the nutritional supplements (or dietary supplements or simply supplements) disclosed herein can be used to inhibit, decrease, or prevent one or more of the DOMS symptoms in mammals and, in particular, in humans.

In another aspect, the present teachings are related to nutritional supplements that may be used to inhibit, decrease, or prevent one or more of the symptoms resulting from a variety of conditions that result in muscle strength loss including, but not limited to, motor neuron diseases (e.g., amyotrophic lateral sclerosis, ALS), muscle wasting in bed-ridden people, muscle atrophy in astronauts, and the like.

In another aspect, the present teachings are related to nutritional supplements that may be used to inhibit, decrease, or prevent one or more of the symptoms resulting from muscle atrophy that occurs following one or more conditions that may result in result in loss of mobility and power, for example: atrophy that occurs with aging (sarcopenia); cerebrovascular accident (stroke); spinal cord injury; peripheral nerve injury (peripheral neuropathy); other injuries; prolonged immobilization; osteoarthritis; rheumatoid arthritis; prolonged corticosteroid therapy; diabetes (diabetic neuropathy); burns; poliomyelitis; Guillain-Barre syndrome; muscular dystrophy; myotonia congenital; myotonic dystrophy; myopathy; fibromyalgia; loss of strength after operations, surgical interventions; and the like.

In yet another aspect, the present teachings are related to nutritional supplements that may be used to inhibit, decrease, or prevent one or more symptoms of muscle damage and/or muscle strength loss that may occur in long distance runners, cyclists, triathletes, swimmers, ultramarathoners, and other athletes during periods of their activity. In this regard, the supplements in accordance with the present teachings may be efficacious for those continuously exercising for an extended period of time such as the exemplified long distance runners, etc. For example, use of the compositions in accordance with the present teachings may improve strength and endurance, which may be beneficial for those seeking such a result. Alternatively or additionally, use of compositions in accordance with the present teachings may inhibit, decrease, or prevent muscle strength loss, which may have the result of maintaining or increasing endurance.

In a further aspect, microarray analysis together with Biodex instrumentation-based measurement of strength (using, e.g., Biodex Multi-Joint system PRO, Biodex Multi-Joint system JPN, and/or Biodex Multi-Joint system w/o CMP, all available from Biodex Medical Systems, Inc., Shirley, N.Y.) provides a new, powerful, and more concrete method of validation for confirmation of physiological outcomes resulting from consumption of nutritional supplement(s).

Compositions

Compositions in accordance with the present teachings may be formulated in an acceptable carrier and may be prepared, packaged, and optionally labeled for modulating the expression of one or more genes, increasing or decreasing the expression of one or more genes, increasing or stimulating DOMS, inhibiting, decreasing, or preventing exercise-induced muscle damage, including but not limited to eccentric exercise-induced muscle damage.

The compositions useful for the practice of the present teachings may be provided in the form of dietary supplements. Representative examples for administering the compositions include but are not limited to providing a daily dose in the form of a tablet, liquid, softgel, gel or mixed with any solution. This dietary supplement can be taken in any form as required.

In some embodiments, a composition for inhibiting, decreasing, or preventing exercise-induced muscle damage may include a combination of at least one adaptogenic, at least one anti-inflammatory agent, and at least one antioxidant. In some embodiments, the adaptogenic is Rhodiola (or Rhodiola extract) and/or Ashwagandha (or Ashwagandha extract); the anti-inflammatory agent is rose hips (or rose hips extract) and/or grape seeds (or grape seeds extract); and the antioxidant is prickly pear (or prickly pear extract) and/or Astaxanthin (or Astaxanthin extract).

In some embodiments, a composition for inhibiting, decreasing, or preventing exercise-induced muscle damage may include a combination of Rhodiola, rose hips, and astaxanthin. Rhodiola may be present as an extract in an amount ranging from about 10 to about 1000 mg, in an amount ranging from about 50 to about 750 mg, in an amount ranging from about 100 to about 500 mg, in an amount of about 200 mg; rose hips may be present as an extract in an amount ranging from about 30 to about 3000 mg, in an amount ranging from about 50 to about 2000 mg, in an amount ranging from about 100 to about 1000 mg, in an amount of about 600 mg; astaxanthin may be present as an extract in an amount ranging from about 0.1 to about 100 mg, in an amount ranging from about 0.5 to about 50 mg, in an amount ranging from about 1 to about 10 mg, in an amount of about 4 mg; wherein the combination inhibits, decreases, or prevents exercise-induced muscle damage in a subject. The composition may include an excipient. The exercise-induced muscle damage may be eccentric exercise-induced muscle damage. The above ranges reflect the daily amounts of combinations of extracts that may be administered to a subject.

In some embodiments, a composition for inhibiting, decreasing, or preventing exercise-induced muscle damage may include a combination of Ashwagandha, grape seeds, and prickly pear. Ashwagandha may be present as an extract in an amount ranging from about 1 to about 2000 mg, in an amount ranging from about 5 to about 1000 mg, in an amount ranging from about 20 to about 500 mg, in an amount of about 125 mg; grape seeds may be present as an extract in an amount ranging from about 10 to about 3000 mg, in an amount ranging from about 10 to about 1000 mg, in an amount ranging from about 30 to about 1000 mg, in an amount ranging from about 100 to about 500 mg, in an amount of about 200 mg; prickly pear may be present as an extract in an amount ranging from about 20 to about 5000 mg, in an amount ranging from about 50 to about 3000 mg, in an amount ranging from about 100 to about 1000 mg, in an amount of about 500 mg; wherein the combination inhibits, decreases, or prevents exercise-induced muscle damage in a subject. The composition may include an excipient. The exercise-induced muscle damage may be eccentric exercise-induced muscle damage. The above ranges reflect the daily amounts of combinations of extracts that may be administered to a subject.

In some embodiments, a composition for inhibiting, decreasing, or preventing DOMS may include a combination of Rhodiola, rose hips, and astaxanthin. Rhodiola may be present as an extract in an amount ranging from about 10 to about 1000 mg, in an amount ranging from about 50 to about 750 mg, in an amount ranging from about 100 to about 500 mg, in an amount of about 200 mg; rose hips may be present as an extract in an amount ranging from about 30 to about 3000 mg, in an amount ranging from about 50 to about 2000 mg, in an amount ranging from about 100 to about 1000 mg, in an amount of about 600 mg; astaxanthin may be present as an extract in an amount ranging from about 0.1 to about 100 mg, in an amount ranging from about 0.5 to about 50 mg, in an amount ranging from about 1 to about 10 mg, in an amount of about 4 mg; wherein the combination inhibits, decreases, or prevents DOMS in a subject. The composition may include an excipient. The above ranges reflect the daily amounts of combinations of extracts that may be administered to a subject.

In some embodiments, a composition for inhibiting, decreasing, or preventing DOMS may include a combination of Ashwagandha, grape seeds, and prickly pear. Ashwagandha may be present as an extract in an amount ranging from about 1 to about 2000 mg, in an amount ranging from about 5 to about 1000 mg, in an amount ranging from about 20 to about 500 mg, in an amount of about 125 mg; grape seeds may be present as an extract in an amount ranging from about 10 to about 3000 mg, in an amount ranging from about 10 to about 1000 mg, in an amount ranging from about 30 to about 1000 mg, in an amount ranging from about 100 to about 500 mg, in an amount of about 200 mg; prickly pear may be present as an extract in an amount ranging from about 20 to about 5000 mg, in an amount ranging from about 50 to about 3000 mg, in an amount ranging from about 100 to about 1000 mg, in an amount of about 500 mg; wherein the combination inhibits, decreases, or prevents DOMS in a subject. The composition may include an excipient. The above ranges reflect the daily amounts of combinations of extracts that may be administered to a subject.

In some embodiments, a composition for modulating the expression of one or more genes that are correlated with exercise-induced muscle damage may include a combination of Rhodiola, rose hips, and astaxanthin. Rhodiola may be present as an extract in an amount ranging from about 10 to about 1000 mg, in an amount ranging from about 50 to about 750 mg, in an amount ranging from about 100 to about 500 mg, in an amount of about 200 mg; rose hips may be present as an extract in an amount ranging from about 30 to about 3000 mg, in an amount ranging from about 50 to about 2000 mg, in an amount ranging from about 100 to about 1000 mg, in an amount of about 600 mg; astaxanthin may be present as an extract in an amount ranging from about 0.1 to about 100 mg, in an amount ranging from about 0.5 to about 50 mg, in an amount ranging from about 1 to about 10 mg, in an amount of about 4 mg; wherein the combination modulates the expression of one or more genes that may be correlated with exercise-induced muscle damage. The composition may include an excipient. The exercise-induced muscle damage may be eccentric exercise-induced muscle damage.

Examples of genes that can be modulated by the compositions in accordance with the present teachings and are correlated with exercise-induced muscle damage include, but are not limited to, PPARα (peroxisome proliferative activated receptor, alpha); PPARδ (peroxisome proliferative activated receptor, delta); IRF5 (Interferon regulatory factor 5); PLAUR (plasminogen activator, urokinase receptor); RSU1 (Ras suppressor protein 1); CEBPD (CCAAT/enhancer binding protein delta); IFI16 (interferon, gamma-inducible protein 16); TNNT2 (troponin-T type 2); GJA1 (gap junction protein, alpha-like); and SCN3B (sodium channel, voltage-gated, type III, beta). The modulation of gene expression may be up-regulation of gene expression. Alternatively, the modulation of gene expression may be down-regulation of gene expression. Gene expression levels may be measured in one or more tissue samples taken from the skeletal muscle of the subject, which muscle is (or was) involved in the exercise. For example, needle biopsies of the vastus lateralis may be taken from the ECC leg and the control leg (for comparison) at 3-4 hours post-exercise.

In some embodiments, a composition for modulating the expression of one or more genes that are correlated with exercise-induced muscle damage may include a combination of Ashwagandha, grape seeds, and prickly pear. Ashwagandha may be present as an extract in an amount ranging from about 1 to about 2000 mg, in an amount ranging from about 5 to about 1000 mg, in an amount ranging from about 20 to about 500 mg, in an amount of about 125 mg; grape seeds may be present as an extract in an amount ranging from about 10 to about 3000 mg, in an amount ranging from about 10 to about 1000 mg, in an amount ranging from about 30 to about 1000 mg, in an amount ranging from about 100 to about 500 mg, in an amount of about 200 mg; prickly pear may be present as an extract in an amount ranging from about 20 to about 5000 mg, in an amount ranging from about 50 to about 3000 mg, in an amount ranging from about 100 to about 1000 mg, in an amount of about 500 mg; wherein the combination modulates the expression of one or more genes that may be correlated with exercise-induced muscle damage. The composition may include an excipient. The exercise-induced muscle damage may be eccentric exercise-induced muscle damage.

Examples of genes that can be modulated by the compositions in accordance with the present teachings and are correlated with exercise-induced muscle damage include, but are not limited to, PPARα (peroxisome proliferative activated receptor, alpha); PPARδ (peroxisome proliferative activated receptor, delta); IRF5 (Interferon regulatory factor 5); PLAUR (plasminogen activator, urokinase receptor); RSU1 (Ras suppressor protein 1); CEBPD (CCAAT/enhancer binding protein delta); IFI16 (interferon, gamma-inducible protein 16); TNNT2 (troponin-T type 2); GJA1 (gap junction protein, alpha-like); and SCN3B (sodium channel, voltage-gated, type III, beta). The modulation of gene expression may be up-regulation of gene expression. Alternatively, the modulation of expression may be down-regulation of gene expression. Gene expression levels may be measured in one or more tissue samples taken from the skeletal muscle of the subject, which muscle is (or was) involved in the exercise. For example, needle biopsies of the vastus lateralis may be taken from the ECC leg and the control leg (for comparison) at 3-4 hours post-exercise.

Modes of Administration

The compositions in accordance with the present teachings may be administered systemically or locally. For systemic use, the compositions are formulated for parenteral (e.g., intravenous, subcutaneous, intramuscular, intraperitoneal, intranasal or transdermal) or enteral (e.g., oral or rectal) delivery according to conventional methods. Intravenous administration can be by a series of injections or by continuous infusion over an extended period. Administration by injection or other routes of discretely spaced administration can be performed at intervals ranging from weekly to once to three times daily. Alternatively, the compositions disclosed herein may be administered in a cyclical manner (administration of disclosed composition; followed by no administration; followed by administration of disclosed composition; and the like). Treatment can continue until the desired outcome is achieved. Alternatively, administration of the compositions may be continual, and thereby be a preventative administration, rather than an administration for treatment.

In general, compositions in accordance with the present teachings may include a cosmetically or pharmaceutically acceptable vehicle, such as saline, buffered saline, 5% dextrose in water, borate-buffered saline containing trace metals or the like. Compositions in accordance with the present teachings may further include one or more excipients, for example, vitamin A, vitamin D, or calcium; preservatives; solubilizers; buffering agents; albumin to prevent protein loss on vial surfaces; lubricants; fillers; stabilizers; and the like. Methods of formulation are well known and are disclosed, for example, in Remington's Pharmaceutical Sciences, Gennaro, Mack Publishing Co., Easton Pa.: 1990, which is incorporated herein by reference.

Compositions for use in accordance with the present teachings may be in the form of sterile, non-pyrogenic liquid solutions or suspensions, coated capsules, suppositories, lyophilized powders, transdermal patches or other forms are known. Local administration may be by injection at the site of injury or defect, or by insertion or attachment of a solid carrier at the site, or by direct, topical application of a viscous liquid, or the like. For local administration, the delivery vehicle may provide a matrix for the growing bone or cartilage, and may be a vehicle that can be absorbed by the subject without adverse effects.

Aqueous suspensions may contain the extract ingredients in accordance with the present teachings in admixture with pharmacologically acceptable excipients such as vitamin A, vitamin D, and calcium, suspending agents, such as methyl cellulose; and wetting agents, such as lecithin, lysolecithin or long-chain fatty alcohols. The aqueous suspensions may also contain preservatives, coloring agents, flavoring agents, sweetening agents and the like in accordance with industry standards.

Compositions in accordance with the present teachings may be orally administered in the form of a pill, tablet, powder, bar, food, beverage, lozenge, and the like. It is to be understood that the compositions in accordance with the present teachings can optionally contain one or more excipents approved for use in pharmaceuticals, nutraceuticals, foodstuffs, and/or dietary supplements. Additionally, compositions in accordance with the present teachings may be presented as a dried or powdered product for reconstitution with water or other suitable vehicle before use. Furthermore, liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, or fractionated vegetable oils); and preservatives (e.g., methyl or propyl-p-hydroxybenzoates or sorbic acid).

When administered in the form of a beverage, compositions in accordance with the present teachings may be water-based, milk-based, tea-based, fruit juice-based, or some combination thereof.

Compositions in accordance with the present teachings may also be orally administered in the form of a solid prepared by conventional means with pharmaceutically acceptable excipients such as binding agents (e.g., pregelatinized maize starch, polyvinyl pyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The solids may be coated by methods well known in the art. The composition in accordance with the present teachings may take the form of a two-piece hard shell capsule, a soft gelatin capsule, or a powder to be dissolved in a liquid for oral consumption. Preparations for oral administration may be suitably formulated to give controlled release of the active compound.

Compositions in accordance with the present teachings that are orally administered may further comprise thickeners, including xanthum gum, carboxymethyl-cellulose, carboxyethyl-cellulose, hydroxypropyl-cellulose, methyl-cellulose, microcrystalline cellulose, starches, dextrins, fermented whey, tofu, maltodextrins, polyols, including sugar alcohols (e.g., sorbitol and mannitol), carbohydrates (e.g., lactose), propylene glycol alginate, gellan gum, guar, pectin, tragacanth gum, gum acacia, locust bean gum, gum arabic, gelatin, as well as mixtures of these thickeners. These thickeners are typically included in the formulations in accordance with the present teachings at levels up to about 0.1%, depending on the particular thickener involved and the viscosity effects desired.

Orally administered compositions in accordance with the present teachings may, and typically will, contain an effective amount of one or more sweeteners, including carbohydrate sweeteners and natural and/or artificial no/low calorie sweeteners. The amount of the sweetener used in the formulations in accordance with the present teachings may vary, but typically depends on the type of sweetener used and the sweetness intensity desired.

In addition to the formulations described previously, the compounds may also be formulated as a sustained and/or timed release formulation. Common timed and/or controlled release delivery systems include, but are not be limited to, starches, osmotic pumps, or gelatin micro capsules.

The compositions may, if desired, be presented in a pack or dispenser device that may comprise one or more unit dosage forms comprising a composition in accordance with the present teachings. The pack may, for example, comprise metal or plastic foil, such as a blister pack. The pack or dispenser device may be accompanied by instructions for administration.

Preparations of compositions in accordance with the present teachings for topical and local application may include aerosol sprays, lotions, gels and ointments in cosmetically or pharmaceutically appropriate vehicles that may comprise lower aliphatic alcohols, polyglycols such as glycerol, polyethylene glycol, esters of fatty acids, oils and fats, and silicones. The preparations may further comprise antioxidants, such as ascorbic acid or tocopherol, and preservatives, such as p-hydroxybenzoic acid esters.

Parenteral preparations may comprise sterile or sterilized products.

Injectable compositions may be provided containing a combination of the extracts in accordance with the present teachings and any of the well-known injectable carriers. These may contain salts for regulating the osmotic pressure.

Other useful dosage forms can be prepared by methods and techniques that will be well understood by those of skill in the art and may include the use of additional ingredients in producing tablets, capsules, or liquid dosage forms. Although exemplary dosages, dose frequencies, and methods of administration are discussed herein, these are merely exemplary and it is to be understood that the dose, dose frequency, and mode of administration may vary according to the age, body weight, condition and response of the individual consumer or patient, and the particular composition in accordance with the present teachings that is used.

### EXAMPLES

### Example 1

### Identification of Genes/Molecular Targets/Pathways Involved in the Body's Response to Exercise

This study examined the effect of two nutritional supplements containing mixtures of plant extracts with adaptogenic, anti-oxidant, and anti-inflammatory properties on changes in gene expression following an eccentric exercise. Eccentric actions cause transient muscle damage. Thirty healthy male subjects 18-30 yrs old were included in this study over a period of 42 days. In Stage 1 of the study, subjects exercised one leg (knee extensors) and a muscle biopsy of both legs (vastus lateralis muscle) was taken at 3-4 hours post-exercise.

In Stage 2 of the study, subjects were randomly assigned to receive one of three treatments (supplement 1, supplement 2, and placebo) over a period of 28 days. Supplement 1 comprised: Rhodiola+Rose hips+Astaxanthin. Supplement 2 comprised: Ashwagandha+Grape Seed+Prickly Pear. The placebo comprised inert excipients and processing aids used for supplements.

In Stage 3 of the study, after 28 days of taking supplements or placebo, subject repeated the exercise routine performed during Stage 1 with the contralateral leg (knee extensors) and a muscle biopsy of both legs was taken at 3-4 hours post-exercise. Subjects continued to take the supplements or placebo during stage 3.

Expression profiling and analysis was performed on the RNA isolated from the biopsied muscle tissue using Agilent Whole Genome Chips (Gene Logic, Gaithersburg, Md.). The array data were analyzed to identify genes that have p-value<0.02. Specifically, ANCOVA (age and BMI covariates) was used to screen the significantly differentially expressed (p<0.02) genes. The profiles of up- and down-regulated genes for supplement 1 vs. placebo and supplement 2 vs. placebo were analyzed for function and network analysis. Function and network analysis were used to group genes into clusters with similar profiles or belonging to the same pathway. Genes identified by array analysis were validated by qRT-PCR. Genes that were altered in expression level were analyzed to determine if they belonged to a specific biochemical pathway.

Compared with the placebo group: (1) supplement 1 significantly up-regulated 300 and down-regulated 4 genes and supplement 2 significantly up-regulated 116 and down-regulated 16 genes; (2) both supplements enhanced expression of the genes and cellular functions that are involved in the inhibition of inflammatory and oxidative stress mechanisms; (3) among the genes modified by both supplements, PPARα (peroxisome proliferative activated receptor, alpha) was identified as an important player in the response to muscle damage.

Therefore, both nutritional supplements influenced expression of anti-inflammatory and anti-oxidative functions related genes which in turn could reduce eccentric exercise-induced muscle damage.

Table 1 shows a summary of the treatments.

TABLE 1
Summary of treatments and doses used

| Treatment | Dose |
|---|---|
| Supplement 1: Containing (per dose/per | 3 tablets (corresponding to |
| day): 600 mg rose hips powdered extract + | 1 dose) in the morning with |
| 200 mg of rhodiola extract + 4 mg of | breakfast |
| astaxanthin + excipient | |
| Supplement 2: Containing (per dose/per | 3 tablets (corresponding to |
| day): 125 mg ashwagandha + 200 mg of | 1 dose) in the morning with |
| grape seed extract + 500 mg of prickly pear | breakfast |
| extract + excipient | |
| Placebo | 3 tablets (corresponding to |
| Containing: excipients only. | 1 dose) in the morning with |
| | breakfast |

Table 2 shows examples of the excipients that were used in the formulations of the nutritional supplements.

TABLE 2
Excipients and their uses

| Excipient | Function |
|---|---|
| Microcrystaline cellulose | Filler Binder |
| Dextrose | Filler |
| Modified Corn starch | Filler |
| Stearic acid | Lubricant |
| Corn starch | Glidant/lubricant |
| Modified cellulose gum | Disintegrant |
| Silicon Dioxide | Glidant/flow aid |
| Coating Materials: Max 2.5% weight gain, | Processing aid |
| Hydroxypropyl Methylcelulose, USP, | |
| Hydroxypropyl Cellulose, FD&C Red | |
| No. 40 Aluminum Lake, Titanium Dioxide, | |
| USP FD&C Blue | |
| No. 2 Aluminum Lake, | |
| Carnauba Wax: 0.001% | |

Subjects took supplements or placebo during stage 2 (28 days) and during stage 3 (7 days) of the study for a total of 35 days. Study supplements were provided as tablets and supplied in bottles. Supplement labels complied with the cGCP label regulations. They supplied no information about the subject, just a number that allowed identification of the study group and tracking of each individual bottle. The storage conditions for the study supplement were described on the supplement label, as well as directions for taking the supplement.

Table 3 shows a list of genes modified by both supplements (p<0.02) in supplement 1 vs. placebo and supplement 2 vs. placebo comparisons. These genes may be used to develop a screening assay for evaluating other candidate compositions for efficacy in up- and/or down-regulation of gene expression in accordance with the present teachings. It is to be understood that the listing of genes shown in Table 3 is merely a representative rather than exhaustive list of suitable genes.

By way of example, a screening assay of candidate compositions may include providing an in vitro muscle cell culture (e.g., of any muscle), an animal model or a human muscle tissue, each of which is then treated with a candidate supplement to assess the effect of the supplement's ingredients on the gene expression pattern corresponding to the genes in Table 3. In some embodiments, the gene expression pattern may be determined by any RNA detecting system, including but not limited to Northern blot, real-time polymerase chain reaction (RT-PCR) and variations thereon, microarray, high throughput assays to measure RNA, and the like. In some embodiments, protein products of these genes can be measured using protein-measuring system including but not limited to proteomics approaches, immunoassays, and the like. In some embodiments, observing the functionalities of the gene products or their protein products can be used to evaluate the effect of a candidate composition. As an example of the latter, the LNPEP gene encodes a zinc-dependent aminopeptidase (metalloexopeptidase) that cleaves vasopressin, oxytocin, lys-bradykinin, met-enkephalin, dynorphin A, and other peptide hormones. Thus, one can screen for the functionality by measuring the cleavage of these hormones or peptides or any protein that is modulated by LNPEP. Alternatively, an increase in expression of LNPEP serves as a surrogate indicator of increased muscle glucose utilization, which could improve muscle utilization.

TABLE 3
Genes modified by both supplements (p < 0.02)

| | Supplement 1 | | Supplement 2 | |
|---|---|---|---|---|
| Gene | vs. Placebo | | vs. Placebo | |
| Symbol | P-value | Fold Change | P-value | Fold Change |
| LNPEP | 0.00390366 | 3.52373 | 0.012361 | 2.82775 |
| IRF5 | 0.00189901 | 3.02696 | 0.009384 | 2.39591 |
| PPARα | 0.0025692 | 2.93335 | 0.003604 | 2.73247 |
| PPARδ | 0.0107482 | 2.2724 | 0.011011 | 2.21813 |
| NEURL | 0.00724553 | 2.1284 | 0.003517 | 2.25142 |
| CES2 | 0.00352019 | 1.85812 | 0.014477 | 1.63506 |
| HADHB | 0.0104125 | 1.74802 | 0.011355 | 1.71045 |
| PLAUR | 0.0154663 | −3.77282 | 0.011778 | −3.86844 |

Next, lists of the top ten genes that were identified in supplement 1 vs. placebo and supplement 2 vs. placebo comparisons were generated. The results are shown in Tables 4 and 5, respectively, for the two comparisons.

TABLE 4
Top 10 modified genes - supplement 1 vs. placebo

| Gene Symbol | P-value | Fold Change |
|---|---|---|
| SLC38A1 | 0.00129098 | 4.688 |
| RSU1 | 0.00055467 | 3.61561 |
| GIPC1 | 0.00261822 | 3.46846 |
| IRF5 | 0.00189901 | 3.02696 |
| TMEFF1 | 0.00262322 | 3.01268 |
| PPARA | 0.0025692 | 2.93335 |
| ABCC4 | 0.00353871 | 2.79742 |
| EXOC8 | 0.00185527 | 2.25069 |
| RNF8 | 0.00107078 | 2.1126 |
| VTI1B | 0.00304865 | 2.11039 |

TABLE 5
Top 10 modified genes - supplement 2 vs. placebo

| Gene Symbol | P-value | Fold Change |
|---|---|---|
| CEBPE | 0.00388953 | 4.80021 |
| DHCR7 | 0.0030603 | 2.88335 |
| PPARA | 0.0036035 | 2.73247 |
| RABIF | 0.00468558 | 2.46673 |
| ACE2 | 0.00392653 | 2.42859 |
| NEURL | 0.00351743 | 2.25142 |
| GABRD | 0.00131807 | 2.1477 |
| TMEM93 | 0.00140448 | 2.07101 |
| GRIA3 | 0.00567922 | −2.88168 |
| TMCO2 | 0.00327591 | −3.96249 |

Supplements 1 and 2 were successful in controlling one of the main symptoms of DOMS, namely loss of muscle strength.

Described below are examples of some genes whose expression is modulated by administration of dietary supplements in accordance with the present teachings, following eccentric exercise.

PPARα (peroxisome proliferative activated receptor, alpha): This gene is up-regulated by both the supplements (supplement 1 and supplement 2). Studies have recently implicated that PPARα is an anti-inflammatory molecule (Bensinger & Tontonoz, 2008). The exact mechanisms underlying its anti-inflammatory properties still remain unclear. One of the several proposed mechanisms suggests that PPARα may directly interact with transcription factors nuclear factor-kB (NF-kB) or inhibit NF-kB by upregulating the expression of an inhibitor of NF-kB or blocking the expression of inflammatory cytokines such as IL-6.

PPARδ (peroxisome proliferative activated receptor, delta): This gene is up-regulated by both supplements. PPARδ is abundantly expressed in skeletal muscle. A recent study showed that GW501516, a PPARδ agonist, reduced the urinary isoprostanes, which are systemic oxidative stress markers (Risérus et al., 2008, Diabetes 57: 332-339). This suggests the potential role of PPARδ as an antioxidant in skeletal muscle. U.S. Patent Application Publication 2008/0187928 is directed to methods for enhancing exercise performance and discloses interactions between PPARδ and exercise-induced kinases.

IRF5 (Interferon regulatory factor 5): This gene is up-regulated by both supplements. IRF5 is a transcription factor that plays a role in diverse biological processes including virus-mediated activation of interferon, cell growth, differentiation, apoptosis, and immune system activity. Activated IRF5 dimerizes and translocates to the nucleus and then binds the promoter sequences of cytokines such as type I interferons, TNF-alpha, IL-6 or IL-12, activating their transcription.

PLAUR (plasminogen activator, urokinase receptor): This gene is down-regulated by both supplements. PLAUR encodes the receptor for urokinase plasminogen activator. The receptor for urokinase plasminogen activator is a glycosylphosphatidylinositol (GPI)-anchored glycoprotein that may facilitate the invasion of inflammatory cells by regulating membrane-associated plasmin activity, thus potentially exerting anti-inflammatory effects.

RSU1 (Ras suppressor protein 1): This gene is up-regulated by supplement 1. It codes for an inhibitor of ras, which is a stress responsive element, and thus, may inhibit stress pathways. Haptoglobin and Hemopexin are plasma acute phase proteins that bind with high-affinity hemoglobin and heme, respectively. They play an important role in the protection against oxidative stress and inflammation. Using cDNA analysis, it was recently identified that RSU1 is one of the top gene candidates that is functionally related to Haptoglobin and/or Hemopexin. This suggests that RSU1 has anti-inflammatory and/or anti-oxidant properties. Supplement 1 increased the expression level of RSU1 and thus enhanced the anti-inflammatory and/or anti-oxidative functions.

Isometric strength tests were also performed. Muscle strength was measured for two muscle groups, the knee extensors, which was the muscle group stressed by the eccentric exercise, and the knee flexors, which did not perform the eccentric exercise. Also, muscle strength was measured in 2 modes: (i) isometric (no movement, like pushing against an immovable wall); and (ii) isokinetic (dynamic movement over the range of motion of the knee) at two speeds (60 degrees per second, a moderate speed, and 180 degrees per section, a fast speed). These measures were taken to assess whether the supplement affected the various types of strength. If the supplement affected all or most of the isometric and isokinetic knee extensor strength measures, one can conclude that it has a powerful effect on muscle function. The knee flexor strength measures were used as a control. Documenting that the supplement did not affect these measures strengthens the interpretation of any beneficial effects noted for the knee extension measures.

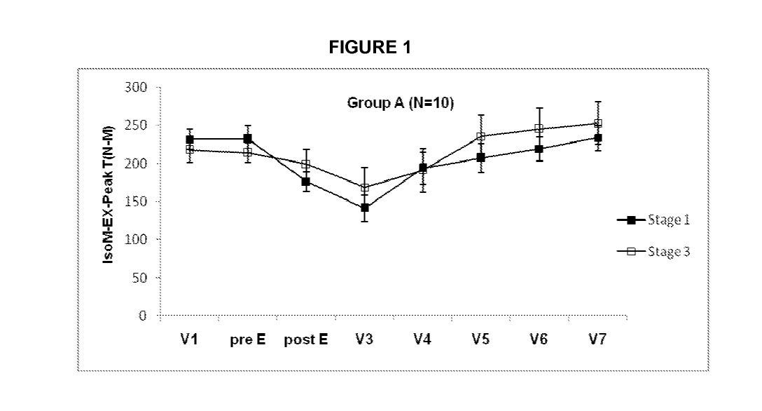

FIGS. 1-3 represent the data for isometric strength, and in particular knee extension data. The results for the other measures, e.g. the knee flexion isometric data, and others, were very similar to the isometric strength measures and are not shown.

FIG. 1 is a graph showing knee extension isometric force for the group receiving the placebo. Stage 1 shows the visit points before taking the placebo. Stage 3 shows the visit points after taking the placebo. Vs represent visits: V1=visit 1, V2=visit 2, etc.

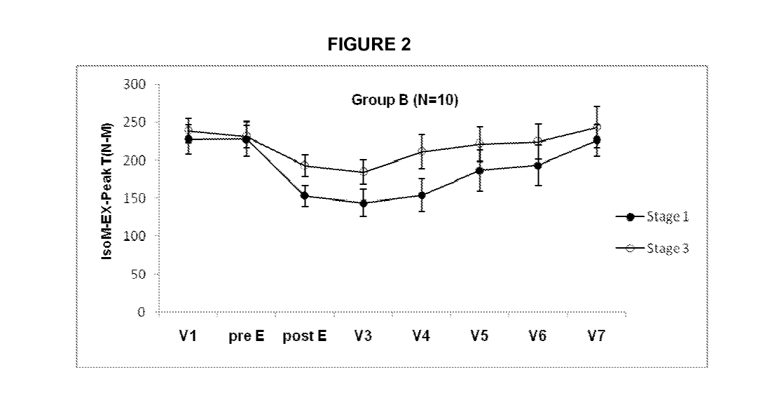

FIG. 2 is a graph showing knee extension isometric force for the group receiving supplement 1. Stage 1 shows the visit points before taking the supplement 1. Stage 3 shows the visit points after taking the supplement 1. Vs represent visits: V1=visit 1, V2=visit 2, etc.

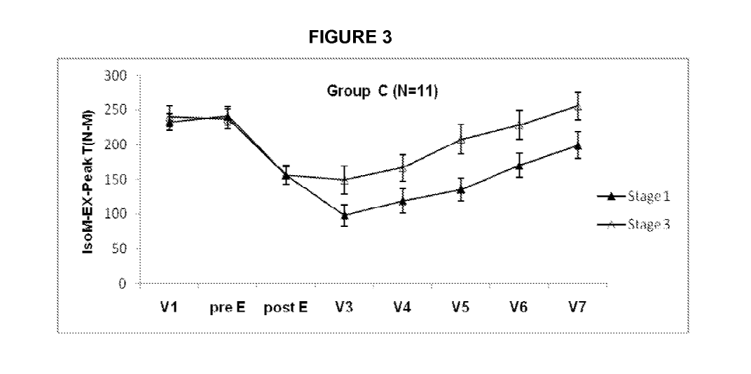

FIG. 3 is a graph showing knee extension isometric force for the group receiving supplement 2. Stage 1 shows the visit points before taking supplement 2. Stage 3 shows the visit points after taking supplement 2. Vs represent visits: V1=visit 1, V2=visit 2, etc.

### Example 2

### Gene Expression of Skeletal Muscle after Eccentric Exercise

Eccentric exercise (ECC) results in muscle damage; however, the molecular mechanisms underlying the damage process remain unclear. Prior microarray studies have examined only a few subjects post-exercise. In this work, microarray technology was used to provide a global analysis of early changes in skeletal muscle gene expression after ECC in a large sample of research subjects.

Thirty healthy men performed 100 contractions on a Biodex Dynamometer® (Biodex Medical Systems, Inc., Shirley, N.Y.). Needle biopsies of the vastus lateralis were taken from the ECC leg and the control leg at 3-4 hrs post-exercise. Samples were frozen in liquid nitrogen, then processed for RNA isolation and microarray data generation at Gene Logic (Cambridge, Mass.). Microarray data analysis was done in Partek Genomics Suite software (Version 6.4), and the outputs were filtered on the extent of change (fold-change (FC) of at least 1.4) and p-value (p=0.005). Genes that met these criteria were then examined to determine the functional pathways affected by ECC.

It was discovered that 463 genes met the above criteria; these genes were then sorted by FC to identify the top 10 differentially expressed genes. The 463 genes were then analyzed and 21 functional groups were found to be associated with these genes. Genes involved in the functions of cell death (n=50) and cellular growth and proliferation (n=51) had the greatest response to ECC. Five of the top 10 differentially expressed genes were located in the functional group of cell death. These 5 genes were all up-regulated: CEBPD (CCAAT/enhancer binding protein (C/EBP), delta), 2.13 FC; IFI16 (interferon, gamma-inducible protein 16), 1.74 FC; TNNT2 (troponin-T type 2), 1.55FC; GJA1 (gap junction protein, alpha-like), 1.51 FC; and SCN3B (sodium channel, voltage-gated, type III, beta), 1.43 FC. The protein product for CEBPD is involved in regulating the inflammatory process. The protein product for IFI16 has many functions with one being the inhibition of cell growth. The protein products for TNNT2, GJA1, and SCN3B all function in regulating ions for the process of excitation-contraction.

Therefore, the alteration in gene expression of the 5 up-regulated genes involved in the cell death function suggests important processes after ECC are inflammatory, growth inhibition, and excitation-contraction.

The data were further analyzed for analysis of effect of supplements on functional pathways and associated genes that were found to be modified as a result of eccentric exercise in the baseline data analysis. The associated genes from these pathways are listed in Table 6.

TABLE 6
Functions of interest - supplements vs. placebo

| | Genes categorized in the function | |
|---|---|---|
| Function | Supplement 1 vs. Placebo | Supplement 2 vs. Placebo |
| Amino Acid | MTHFR, ARG2, SLC38A1, | IGF1, SECISBP2 |
| Metabolism | MTR | |
| Carbohydrate | PPARA, MTOR, LNPEP, | AKR7A2, PPARA, ALDH2, |
| Metabolism | ABCB10, PDPK1, PLAUR, | SORD, LNPEP, SLC2A8, |
| | PPM1A | IGF1, SORBS1, CTBS, |
| | | PLAUR, DCXR, B3GAT2 |
| Cell Cycle | PIAS2, CREM, PA2G4, LIG4, | PPARA, MAP2K6, IGF1, |
| | NF2, PRPF4, GOLGA2, | PPARD, PLAUR, BID, |
| | RECQL, TBRG1 (includes | FANCL |
| | EG: 84897), MTOR, PPM1A, | |
| | TNFSF15, POLK, MXI1, | |
| | MTCP1, MITF, COPS5, | |
| | PLAUR, IRF5, MLL, TERF2, | |
| | CUL2, HAVCR2, PKMYT1, | |
| | MXD4 | |
| Cell Death | PDPK1, MX1 | CA9, MAP2K6, IGF1, |
| | | PPARD, PLAUR, BID, |
| | | MEIS1, IRF5, CEBPE |
| Cell | PPARA, PPID, HTATIP2, | PPARA, ZFYVE9, KCNE2, |
| Morphology | CREM, MITF, RIT1, ACOX1, | NRP2, PLA2G12A, PPARD, |
| | AP1S2, NF2, PLAUR, | PLAUR, MEIS1, SGCA, HPN, |
| | PDPK1, IRF5, RAP1A, MLL, | CEBPE, F11R, PSD, IGF1, |
| | MCF2L, SH3D19, MTOR, | SORBS1, BID, GNRHR |
| | PRDX3, MERTK, CYCS | |
| | (includes EG: 54205), MXD4, | |
| | POLK, MXI1 | |
| Cell Signaling | PPID, GNAI3, MTOR, | TBCA |
| | ABCC4, CASQ2 | |
| Cell-To-Cell | MERTK, PLAUR, RSU1 | MAP2K6, NRP2, IGF1, |
| Signaling and | | PLAUR |
| Interaction | | |
| Cellular | PPARA, NEURL, ACOX1, | PPARA, ZFYVE9, NEURL, |
| Assembly and | AP1S2, LIG4, VTI1B, | ARHGEF12, NRP2, PPARD, |
| Organization | CABIN1, RNF8, TERF2, | PACSIN1, CNP, PLAUR, |
| | PRDX3, MERTK, CD244, | CLASP2, SGCA, GNG7, TST, |
| | CYCS (includes EG: 54205), | DHCR7, IGF1, PSD, |
| | TCP1, MTMR2 | SORBS1, DIAPH2, BID, |
| | | ARHGEF9, NPC1L1, GNRHR |
| Cellular | LTB4R, MTOR, TIMM8A, | IGF1, BID, CLASP2, TST |
| Compromise | PLAUR | |
| Cellular | PPARA, RPH3AL, RAP1A, | PPARA, NEURL, PSD, IGF1, |
| Function and | CABIN1, GNAI3, BNIP3 | SORBS1, PLAUR, |
| Maintenance | (includes EG: 664), MTOR, | ARHGEF9, NPC1L1 |
| | EXOC8, MERTK, CD244, | |
| | HAVCR2, CYCS (includes | |
| | EG: 54205), RSU1 | |
| Cellular Growth | EIF1AY, DCBLD2, PPARA, | PPARA, CA9, SLC2A8, IGF1, |
| and | NEURL, CREM, PIAS2, | PPARD, CNP, PLAUR, BID, |
| Proliferation | PA2G4, RPH3AL, LIG4, | MEIS1, GNG7, HPN |
| | TFR2, LRP1B, NF2, PDPK1, | |
| | CES2 (includes EG: 8824), | |
| | GOLGA2, SET, BNIP3 | |
| | (includes EG: 664), HADHB, | |
| | MTOR, TMEFF1, TCP1, | |
| | PPM1A, BCCIP, MXI1, DAP, | |
| | PREB, MITF, PPARD, | |
| | PLAUR, MX1, PTPN3, | |
| | SCAMP4, MLL, TAF9B, | |
| | CD244, ANAPC5, RSL1D1, | |
| | HAVCR2, ALCAM, YME1L1, | |
| | PPAT, NRP1 | |
| Cellular | PTP4A1, PDPK1, ALCAM, | F11R, NRP2, IGF1, PLAUR, |
| Movement | TNFSF15, PARP9, RAP1A, | HPN |
| | MLL, NRP1 | |
| DNA | TERF2, ERCC8, MTCP1, | None |
| Replication, | LIG4, COPS5, DHX36, POLK, | |
| Recombination, | ALKBH3, SET, NUDT15, | |
| and Repair | MLL, RECQL | |
| Free Radical | CYCS (includes EG: 54205) | None |
| Scavenging | | |
| Gene | PPARA, TRIP11, MLXIPL, | PPARA, IGF1, PPARD, |
| Expression | HTATIP2, CREM, PIAS2, | CEBPE |
| | PA2G4, MITF, PPARD, | |
| | RNF8, MLX, TEAD1, MTOR, | |
| | MTERF, MXI1 | |
| Lipid | PPARA, MLXIPL, ARV1, | PPARA, HADHB, DHCR7, |
| Metabolism | PPARD, ACOX1, MX1, | IGF1, AKR1C3, PPARD, BID, |
| | HADHB, MTOR, LTB4R, | LSS, PNPLA2, PNPLA4, |
| | INSIG2, DLAT, CYCS | NPC1L1 |
| | (includes EG: 54205), MTMR2, | |
| | SMG1 | |
| Molecular | PPARA, ABCB10, PPARD, | PPARA, PPARD, PLAUR, |
| Transport | MTHFR, ACOX1, PLAUR, | PNPLA2, GNG7, LNPEP, |
| | PDPK1, SLC38A1, MTR, | SLC2A8, SLC9A7, IGF1, |
| | GNAI3, MTOR, INSIG2, | PSD, SORBS1, BID, |
| | LNPEP, PPM1A, ABCC4, | PNPLA4, GRIA3, NPC1L1 |
| | CASQ2 | |
| Nucleic Acid | ADSS, PPARA, GNAI3, | PSD, IGF1, CNP, BID, |
| Metabolism | GMPS, ACOX1, DLAT, | DCXR, GNG7 |
| | DCTD, ABCC4, NUDT15, | |
| | PPAT | |
| Protein | PPARA, MTOR, LNPEP, | PPARA |
| degradation | CUL2, ANAPC5, USP33, | |
| | RNF6 | |
| Protein folding | TCP1 | TBCA |
| Protein | PPARA, TBRG1 (includes | PPARA, IGF1, NPC1L1 |
| Synthesis | EG: 84897), MTOR, LNPEP, | |
| | CUL2, GIPC1, ANAPC5, | |
| | HPS4, USP33, RNF6 | |
| Protein | MTOR, LTBP2, GIPC1, | None |
| Trafficking | SYNJ2BP, AP1S2, HPS4, | |
| | TIMM8A | |
| Small Molecule | PPARA, MLXIPL, ABCB10, | PPARA, AKR1C3, PPARD, |
| Biochemistry | ARV1, PDPK1, SLC38A1, | CNP, PLAUR, CES2 |
| | ARG2, CES2 (includes | (includes EG: 8824), DCXR, |
| | EG: 8824), MTR, HADHB, | PNPLA2, SECISBP2, GNG7, |
| | LTB4R, MTOR, INSIG2, | TST, HADHB, SORD, |
| | LNPEP, PPM1A, MTMR2, | DHCR7, SLC2A8, LNPEP, |
| | SMG1, PPARD, MTHFR, | IGF1, PSD, CTBS, BID, LSS, |
| | ACOX1, PLAUR, MX1, DCTD, | PNPLA4, NPC1L1 |
| | NUDT15, ADSS, TMLHE, | |
| | GNAI3, GMPS, DLAT, CYCS | |
| | (includes EG: 54205), ABCC4, | |
| | PPAT | |
| Vitamin and | PPID, PLAUR, CASQ2 | IGF1, PLAUR |
| Mineral | | |
| Metabolism | | |

### Example 3

### Two Dietary Supplements with Antioxidant and Anti-Inflammatory Properties Protect against Eccentric Exercise-Induced Strength Loss

Eccentric actions cause transient muscle damage. Symptoms of exertional muscle damage are muscle soreness, swelling, stiffness and a prolonged loss of strength. Mechanical damage to the muscle also results in generation of inflammatory cytokines that produce secondary damage. This study investigated whether two dietary supplements with antioxidant and anti-inflammatory properties could attenuate the effects of eccentric exercise-induced muscle damage.

Thirty-one healthy men aged 18-30 were randomly assigned to receive a formula containing the placebo or 1 of 2 dietary supplements for 35 days. The study period was composed of three stages: Stage 1—subjects exercised knee extensors of one leg and a muscle biopsy of both legs (data not reported here) was taken at 3-4 hours post-exercise; Stage 2—subjects took a dietary supplement or placebo for a 28-day period; Stage 3—subjects repeated the exercise with the contralateral leg and a muscle biopsy of both legs was taken at 3-4 hours post-exercise. Subjects continued to take the supplements or placebo during Stage 3. Serum creatine kinase (CK) activity, muscle soreness and muscle strength were measured pre-exercise and each day for 5 days after exercise.

A repeated measures ANOVA indicated that there was temporary muscle damage as evidenced by a significant increase in creatine kinase (CK) activity and muscle soreness and a decrease in strength (p<0.0001) post-exercise. Analysis of the Stage 1 vs Stage 3 showed a significant attenuation of strength loss post-supplement for Supplement 1(p=0.0205) and Supplement 2 groups (p=0.0038), but not for the Placebo group (p=0.2205). The effects of the supplements on CK response and muscle soreness were inconclusive.

Therefore, four weeks of supplementation with either of two dietary supplements with adaptogenic, anti-oxidant, and anti-inflammatory properties reduced strength loss and/or enhanced recovery of strength following an eccentric exercise of the quadriceps.

### Example 4

### The Effects of Two Dietary Supplements on Gene Expression following Eccentric Exercise

Eccentric actions cause transient muscle damage. The cellular and molecular mechanisms underlying the damage process are not well understood. Several studies have reported that antioxidant supplements attenuate exercise-induced muscle injury and oxidative stress and can reduce evidence of damage after eccentric exercise.

This study examined the effect of two dietary supplements containing mixtures of plant extracts with anti-oxidant and anti-inflammatory properties on changes in gene expression following an eccentric exercise. Thirty healthy men aged 18-30 yrs were randomly assigned to receive a formula containing the placebo or 1 of 2 dietary supplements for 35 days. The study period consisted of three stages: Stage 1—subjects exercised one leg (knee extensors) and a muscle biopsy of both legs (vastus lateralis muscle) was taken at 3-4 hours post-exercise; Stage 2—subjects took a dietary supplement or placebo for a 28-day period; Stage 3—subjects repeated the exercise with the contralateral leg and a muscle biopsy of both legs was taken at 3-4 hours post-exercise. Expression profiling was performed using Agilent Whole Genome Chips (Gene Logic, Gaithersburg, Md.). ANCOVA (age and BMI covariates) was used to screen the significantly differentially expressed (p<0.02) genes. The profiles of up- and down-regulated genes for supplement 1 vs. placebo and supplement 2 vs. placebo were analyzed for function and network analysis.

Compared with the placebo group, supplement 1 significantly up-regulated 300 and down-regulated 4 genes and supplement 2 significantly up-regulated 116 and down-regulated 16 genes. Furthermore, both supplements enhanced expression of the genes and cellular functions that are involved in the inhibition of inflammatory and oxidative stress mechanisms. Among the genes modified by both supplements, PPARα (peroxisome proliferative activated receptor, alpha) was identified as an important player in the response to muscle damage.

Therefore, both dietary supplements tested influenced expression of anti-inflammatory and anti-oxidative functions-related genes, which in turn could reduce eccentric exercise-induced muscle damage.

The foregoing detailed description and examples have been provided soley by way of explanation and illustration, and are not intended to limit the scope of the appended claims. Many variations in the embodiments illustrated herein will be apparent to one of ordinary skill in the art, and remain within the scope of the appended claims and their equivalents. Unless incorporated hereinabove, all publications, patents, and patent applications cited herein are hereby incorporated by reference in their entireties.

1. A composition comprising:
an adaptogenic agent selected from the group consisting of a Rhodiola extract, an Ashwagandha extract, and a combination thereof;
an anti-inflammation agent selected from the group consisting of a rose hips extract, a grape seed extract, and a combination thereof; and
an anti-oxidant selected from the group consisting of an astaxanthin extract, prickly pear extract, and a combination thereof.

2. The composition of claim 1, comprising about 10 to about 1000 mg of Rhodiola extract, about 30 to about 3000 mg of rose hips extract, and about 0.1 to about 100 mg of astaxanthin extract.

3. The composition of claim 1, comprising about 200 mg of Rhodiola extract, about 600 mg of rose hips extract, and about 4 mg of astaxanthin extract.

4. The composition of claim 1, comprising about 1 to about 2000 mg of Ashwagandha extract, about 10 to about 3000 mg of grape seed extract, and about 20 to about 5000 mg of prickly pear extract.

5. The composition of claim 1, comprising about 125 mg of Ashwagandha extract, about 200 mg of grape seed extract, and about 500 mg of prickly pear extract.

6. The composition of claim 1, wherein the composition is configured for modulating the expression of one or more genes selected from the group consisting of: PPARα (peroxisome proliferative activated receptor, alpha); PPARδ (peroxisome proliferative activated receptor, delta); IRF5 (Interferon regulatory factor 5); PLAUR (plasminogen activator, urokinase receptor); RSU1 (Ras suppressor protein 1); CEBPD (CCAAT/enhancer binding protein delta); IFI16 (interferon, gamma-inducible protein 16); TNNT2 (troponin-T type 2); GJA1 (gap junction protein, alpha-like); and SCN3B (sodium channel, voltage-gated, type III, beta).

7. The composition of any one of claims 1-5 further comprising an excipient, wherein the composition is configured to inhibit, decrease, or prevent eccentric exercise-induced muscle damage in a subject.

8. A method of inhibiting, decreasing, or preventing a symptom of delayed onset muscle soreness (DOMS) in a subject, comprising administering to the subject the composition of claim 1.

9. A method of inhibiting, decreasing, or preventing exercise-induced muscle damage in a subject, comprising administering to the subject the composition of claim 1.

10. The method of claim 9, wherein the exercise-induced muscle damage is eccentric exercise-induced muscle damage.

11. A dietary supplement for modulating the expression of one or more genes selected from the group consisting of: PPARα (peroxisome proliferative activated receptor, alpha); PPARδ (peroxisome proliferative activated receptor, delta); IRF5 (Interferon regulatory factor 5); PLAUR (plasminogen activator, urokinase receptor); RSU1 (Ras suppressor protein 1); CEBPD (CCAAT/enhancer binding protein delta); IFI16 (interferon, gamma-inducible protein 16); TNNT2 (troponin-T type 2); GJA1 (gap junction protein, alpha-like); and SCN3B (sodium channel, voltage-gated, type III, beta), the dietary supplement comprising an excipient and a combination of: (i) an adaptogenic agent selected from the group consisting of Rhodiola extract, Ashwagandha extract, and a combination thereof; (ii) an anti-inflammation agent selected from the group consisting of rose hips extract, grape seed extract, and a combination thereof; and (iii) an anti-oxidant selected from the group consisting of astaxanthin extract, prickly pear extract, and a combination thereof.

12. The dietary supplement of claim 11, comprising about 10-1000 mg of Rhodiola extract, about 30 to about 3000 mg of rose hips extract, and about 0.1 to about 100 mg of astaxanthin extract.

13. The dietary supplement of claim 11, comprising about 200 mg of Rhodiola extract, about 600 mg of rose hips extract, and about 4 mg of astaxanthin extract.

14. The dietary supplement of claim 11, comprising about 1 to about 2000 mg of Ashwagandha extract, about 10 to about 3000 mg of grape seed extract, and about 20 to about 5000 mg of prickly pear extract.

15. The dietary supplement of claim 11, comprising about 125 mg of Ashwagandha extract, about 200 mg of grape seed extract, and about 500 mg of prickly pear extract.

16. A method of modulation of the expression of one or more genes correlated with eccentric exercise-induced muscle damage in a subject, comprising administering the dietary supplement of any one of claims 11-15 to the subject.

17. The method of claim 16 wherein the modulation is up-regulation of gene expression.

18. The method of claim 16 wherein the modulation is down-regulation of gene expression.
